(19) United States
(12) Patent Application Publication
(10) Pub. No.: US 2022/0390401 A1
(43) Pub. Date: Dec. 8, 2022

(54) CHEMICAL SENSOR UTILIZING ELECTROCHEMICAL IMPEDANCE SPECTROSCOPY

(71) Applicants: International Business Machines Corporation, Armonk, NY (US); Università del Salento, Lecce (IT)

(72) Inventors: Tim Erdmann, Campbell, CA (US); Angelo Milone, Scorrano (IT); Linda Karin Sundberg, Los Gatos, CA (US); Siddhartha Das, San Mateo, CA (US); Ronald R. Labby, San Jose, CA (US); Giuseppe Maruccio, Scorrano (IT); Krystelle Lionti, San Jose, CA (US); Andrea Fasoli, San Jose, CA (US); Luisa Dominica Bozano, Los Gatos, CA (US); Maria S. Chiriacò, Lecce (IT)

(21) Appl. No.: 17/316,877

(22) Filed: May 11, 2021

(51) Int. Cl. G01N 27/02; G01N 33/00

(52) U.S. Cl. CPC G01N 27/026; G01N 33/0047

(57) ABSTRACT

A method of preparing electronically conductive polyaniline that forms a self-supporting dispersion in water is described. The binder-free dispersion was coated on a pair of interdigitated metal electrodes to form a gas sensing layer of a chemical sensor. The chemical sensor utilizes electrochemical impedance spectroscopy (EIS) to detect and characterize a chemical compound in a gaseous state in contact with the sensing layer. Impedance of the sensing layer is measured over a range of alternating current frequencies. The impedance data allows identification and concentration of the chemical compound to be determined when compared to reference impedance data. The analysis of the impedance measurements is adaptable to machine learning.

### BACKGROUND

The present invention relates to a chemical sensor utilizing electrochemical impedance spectroscopy, and more specifically, to a gas sensor employing a binder-free conductive polymer sensing layer.

Sensing of volatile organic compounds (VOCs) is needed for numerous applications such as, for example, evaluating indoor air quality, monitoring industrial processes, ensuring food and environmental safety, and diagnosing medical conditions. The quality in a given circumstance (e.g., indoor air quality) is determined by the type of VOCs present and their concentrations in the gas phase.

Sensing techniques that utilize chemiresistors, metal-oxide semiconductor sensors, surface acoustic wave sensors, quartz crystal microbalances, microcantilevers, electrochemical sensors, photoionization detectors, and optical sensors allow extraction of one measured value, making the differentiation of VOCs difficult.

Therefore, sensors are needed having appropriate sensitivity to detect the VOCs and the selectivity to differentiate between various VOCs.

### SUMMARY

Accordingly, a method is disclosed, comprising:
providing an initial mixture comprising a polymerizable aromatic amine monomer, an acid, and an aqueous solvent;
adding to the initial mixture an aqueous solution of KHSO₅, KHSO₄, and K₂SO₄, and optionally a co-oxidant, thereby forming a second mixture comprising an initial polymer;
removing any excess salt and monomer from the initial polymer, thereby forming fibers of an electrically conductive polyamine; and
suspending the conductive polyamine in an aqueous solvent, thereby forming a self-stabilized liquid dispersion of the conductive polyamine.

Also disclosed is a chemical sensor, comprising:
a substrate comprising a pair of interdigitated electrodes;
an electrically conductive sensing layer for sensing a chemical compound, the sensing layer disposed on the pair of interdigitated electrodes; and
an impedance analyzer in electrical communication with the sensing layer and interdigitated electrodes;
wherein
the sensing layer comprises fibers of an electrically conductive polyamine in an amount of 98-100 wt % based on total weight of the sensing layer, and
the chemical sensor uses electrochemical impedance spectroscopy to characterize a chemical compound in contact with the sensing layer.

Further disclosed is a method, comprising:
detecting contact of a chemical compound with a sensing layer of a chemical sensor using electrochemical impedance spectroscopy (EIS), the chemical sensor comprising a pair of interdigitated electrodes, the sensing layer comprising fibers of an electrically conductive polyamine in an amount of 98-100 wt % based on total weight of the sensing layer, the conductive polyamine disposed on the interdigitated electrodes, the chemical sensor comprising an EIS analyzer for measuring impedance of the sensing layer at different alternating current frequencies; and
comparing the measured impedance to reference impedance data, thereby identifying the chemical compound and determining a concentration of the chemical compound.

Additionally disclosed is method of making polyaniline fibers, comprising:
mixing an aniline monomer, an aqueous solvent, KHSO₅, KHSO₄, and K₂SO₄ together, thereby forming an aqueous solution comprising polyaniline nanofibers, the aqueous solution having a pH value of less than 8, the method being performed at a temperature between −5° C. and 110° C.

Also disclosed is an electrochemical impedance spectroscopy sensor for volatile organic compounds, comprising:
a substrate having multiple pairs of electrodes thereon; and
conductive polymer nanofibers in contact with the electrodes, the nanofibers made using the above-described method.

Further disclosed is a method, comprising:
using the above-described sensor to obtain electrochemical impedance spectroscopy measurements, thereby identifying an analyte of interest.

The above-described and other features and advantages of the present invention will be appreciated and understood by those skilled in the art from the following detailed description, drawings, and appended claims.

### BRIEF DESCRIPTION OF THE SEVERAL VIEWS OF THE DRAWINGS

FIG. 1A is a schematic overhead drawing of the disclosed sensor.

FIG. 1B is a scanning electron micrograph (SEM) image of polyaniline (PAni) fibers prepared using OXONE™.

FIG. 2A is a photograph of a portion of an actual sensor showing a section of a conductive PAni layer.

FIG. 2B is an optical microscope image showing a second sensor comprising a PAni layer generated using ammonium peroxydisulfate (APS).

FIG. 3 is an optical microscope image of a portion of a sensor chip comprising 32 sensors.

FIG. 4 is a diagram for a prototype sensing device for detecting a compound in a gaseous state.

FIG. 5 is a graph showing an ideal Nyquist plot corresponding to a single gas concentration.

FIG. 6 is a graph showing a Bode plot corresponding to a single gas concentration, where the x-axis is the frequency and the y-axis is the imaginary part of the impedance, ImZ.

FIG. 7 is a graph showing a family of Nyquist plots obtained for different concentrations (ppm) of toluene vapor using the disclosed PAni sensor.

FIG. 8 is a graph showing the corresponding Bode plots for the different concentrations of toluene of FIG. 7.

FIGS. 9 and 10 are graphs showing the percentage change in impedance, Sᵣ(%), and the percentage change in resonance frequency, S_{f}(%), respectively, as a function of toluene concentration in ppm.

FIGS. 11 and 12 compare Sᵣ(%) and S_{f}(%), respectively, of water, 1-propanol, 2-butanone, 2-propanol, acetone, ethanol, ethyl acetate, methanol, octane, toluene, and cyclohexane.

FIG. 13 is a graph depicting the linear fit of the Sᵣ(%) data points at the six lowest concentrations of toluene of FIG. 11. This line has a slope (sensitivity) of -0.02976%/ppm.

FIG. 14 is a bar graph showing the normalized sensitivities of the sensor to the compounds of FIG. 11.

FIG. 15 is a block diagram showing an example of a structure of a computer system and computer program code that can be used to automatically and programmatically implement the processes of the invention.

FIG. 16 is a photograph of the coating apparatus used to coat the disclosed dispersion.

### DETAILED DESCRIPTION

Disclosed is a chemical sensor utilizing electrochemical impedance spectroscopy (EIS) to detect chemical compounds, more specifically compounds in a gaseous state (e.g., water vapor, volatile organic compounds, ammonia, hydrogen, hydrogen sulfide, methane, hydrogen peroxide, carbon dioxide, nitric acid, hydrochloric acid, phosphoric acid, phosgene). The chemical compounds to be detected are referred to herein simply as “analytes.” The sensor comprises i) a pair of interdigitated metal electrodes disposed on a substrate and ii) a conductive layer (also referred to herein as the “sensing layer”) comprising a binder-free conductive polymer disposed on the electrodes. The effective operating temperature of the sensor is about −100° C. to about 300° C.

The sensor operates under excitation with a sinusoidal alternating current (AC) or voltage close to its resonant frequency but (using an impedance analyzer) can be characterized in a larger frequency range (e.g., from about 20 Hz to about 20 MHz) to analyze the impedance of the sensing layer when contacted by one or more analytes. A direct current or voltage (DC) can be applied to tune the working conditions. From the impedance data, the identity and concentration of one or more analytes can be determined. Impedance (Z) is a complex number having a real component (ReZ) and an imaginary component (ImZ), where ReZ is the resistive component of Z, and ImZ is the reactive (i.e., capacitive/inductive) component of Z. Beyond this, impedance spectroscopy measures also the frequency dependent phase shift and change in the modulus of Z (|Z|). The impedance changes as a function of the AC frequency, analyte concentration, electrostatic interactions of the sensing layer with an analyte, and/or changes in the morphology of the sensing layer caused by the analyte. The analyzed data are compared to reference impedance data of various analytes stored in a computer database to determine the identity and concentration of the analyte. The process of identifying chemical compounds by EIS using the disclosed chemical sensor is adaptable to machine learning.

Further disclosed is a sensing device comprising one or more of the chemical sensors, where the device is capable of measuring the impedance data used to determine the identity and concentration of one or more analytes. As one non-limiting example, the device can be a self-contained device, in which all components for detecting, identifying and determining the identity and concentration of an analyte are contained in a handheld device, including the chemical sensor, impedance analyzer, computer, data storage, user interface, software, display, and reference impedance data of analytes of interest. Alternatively, the chemical sensor can be a portion of a remote sensing device, which communicates remotely generated impedance data to a central computing facility for further analysis and/or storage of the data. In this instance, the computing facility can house the computer hardware, computer software, storage media, and reference impedance data used to analyze, identify and characterize an analyte, thereby allowing miniaturization of the remote sensing device. Two or more remote sensor devices at the same or different locations can be networked to a central computing and data storage facility. Communication can occur by way of a wired network or a wireless network (e.g., radio frequency local area network (i.e., wifi), mobile cellular network, and/or satellite network). The computing and data storage facility can be a shared cloud facility or a private network.

The sensing layer comprises a polymer, preferably in the form of a network of fibers. Analytes can diffuse to the interior of the fibers and/or adsorb to the surface of the fibers. The sensitivity of the sensor is related to the amount of surface area of the fibers available for contact with an analyte. Selectivity of the sensor is related to the nature of the interactions of the analyte with the fibers (i.e., changes in the electronic and morphological properties of the sensing layer and the change(s) to these properties induced by contact with an analyte).

In a preferred embodiment, the sensing layer consists essentially of conductive polyamine (i.e., 98-100 wt % polyamine based on total weight of the sensing layer). More specifically, the sensing layer excludes a non-conductive additive serving as a binder or matrix for the conductive polyamine. By contrast, previously reported sensors employ electrospun polymer blend/composite nanofibers, which contain a conductive material (e.g., carbon black, carbon nanotubes, polyaniline) dispersed in a non-conductive polymer binder (e.g., poly(methyl methacrylate), PMMA). Preferably the conductive polyamine is an electrically conductive form of polyaniline, referred to herein as PAni, or an electronically conductive polyamine of a “substituted aniline”. A substituted aniline has at least one substituent other than hydrogen at aromatic ring positions 2, 3, 5, and/or 6, and/or at the nitrogen of aniline. These ring positions are labeled in the following chemical structure of aniline. Preferred substituents include C₁-C₁₀ alkyl (e.g., methyl, ethyl, propyl) or cycloalkyl groups.

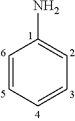

The disclosed sensing layer is not a composite comprising PAni and non-conductive binder. Moreover, the PAni of the disclosed sensing layer is not electrospun, which simplifies production of the sensing layer. The disclosed sensing layer also has greater available surface area for contacting an analyte compared to previous PAni-containing sensing layers due to the high density of fibers per unit area of the sensor and the absence of a binder.

The PAni is prepared by oxidation of aniline in aqueous acid (e.g., hydrochloric acid) according to the reaction below.

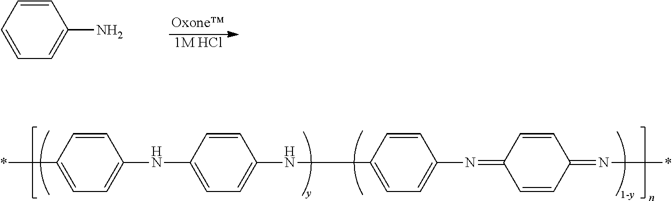

where n is a number having an average value greater than one, and y is a number wherein 0≤y<1. Preferably, y is much less than 1, most preferably y is 0. More specifically, PAni can comprise repeat units selected from the group consisting of free base (leucoemeraldine, y=1), partially oxidized base (emeraldine, 0<y<1), fully oxidized base (pernigranaline, y=0), partially protonated forms of any of the foregoing, fully protonated forms of any of the foregoing, and combinations of any of the foregoing, with the proviso that the PAni comprises at least one diimine group selected from the group consisting of.

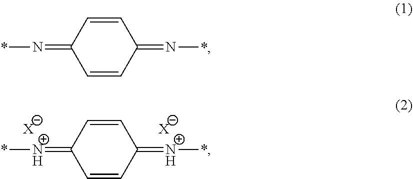

and combinations thereof, wherein each X^{⊖} is an independent negative-charged counterion. Each X^{⊖} can be the same or a different counterion. Exemplary non-limiting X^{⊖} groups include chloride, bromide, iodide, sulfate, sulfite, bisulfite (hydrogen sulfite), bisulfate (hydrogen sulfate), acetate, hydroxide, carbonate, and bicarbonate (hydrogen carbonate).

Oxidized and protonated forms of the repeat units of PAni are illustrated below.

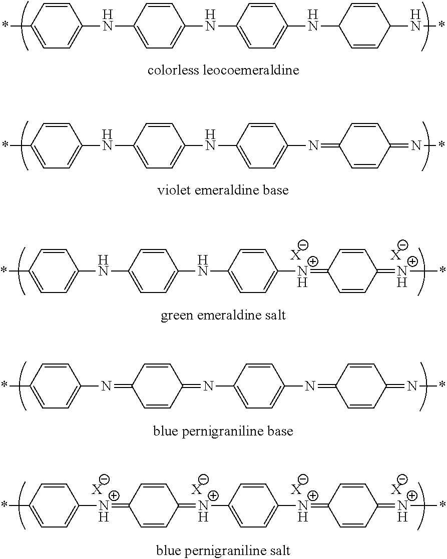

Also disclosed is a method of preparing a stable liquid dispersion of a conductive fibrous polyamine using OXONE™, also known as potassium peroxymonosulfate, trademark owned by Lanxess Deutschland GMBH. OXONE™ comprises KHSO₅, .KHSO₄,. and K₂SO₄ in a 1:0.5:0.5 molar ratio. The method comprises i) forming an initial mixture comprising water, an acid, (e.g., HCl), and a polymerizable aromatic amine monomer, ii) adding an aqueous solution comprising OXONE™ and, optionally, a co-oxidant (e.g., sodium hypochlorite (NaOCl)), thereby forming a second mixture comprising an initial polyamine by oxidative polymerization of the aromatic amine monomer, iii) removing any excess salts and monomer from the initial polymer, thereby forming an electrically conductive polyamine, and iv) suspending the electrically conductive polyamine in an aqueous solvent, thereby forming a self-stabilized homogeneous dispersion of the electrically conductive polyamine. Preferably, the polymerizable aromatic amine monomer is aniline, the electrically conductive polymer is an electrically conductive form of polyaniline (PAni), and the dispersion contains fibers of PAni. The pH of the initial mixture is <8, more specifically between 0 and 6. The pH of the final liquid dispersion is <8, more specifically between 0 and 6. The oxidative polymerization can be performed at a reaction temperature between −5° C. and 110° C. at 1 atm pressure, more preferably at a reaction temperature in the range of 15° C. to 40° C. at 1 atm pressure, most preferably at a reaction temperature of 18° C. to 25° C. at 1 atm pressure. The oxidative polymerization using OXONE can be performed with or without agitation.

The initial polymer can be purified to remove excess salts using known methods (e.g., by centrifugation, dialysis, and/or filtration). The resulting “wet” conductive polyamine can then be re-dispersed in water or an aqueous mixture of water and organic solvent to yield a stable coatable liquid dispersion. The liquid dispersion comprises the PAni in an amount of between 0 wt % and 4 wt % based on total weight of the dispersion, more preferably in an amount greater than 0 wt % and less than or equal to 1 wt % based on total weight of the dispersion.

The dispersion is stable for at least 24 hours, more specifically at least 48 hours, and is compatible with known liquid coating techniques (e.g., spin coating, dip coating, blade coating, spray coating) used to form a coated film layer. Removal of solvent from an initial coated layer yields an electrically conductive sensing layer. The sensing layer prepared with PAni comprises a dense porous network of colored (deep green to blue-black) fibers. The PAni fibers can have an average circular cross-sectional diameter of about between 1 nm and 500 nm, more particularly 20 nm to about 150 nm, and even more particularly 20 nm to 100 nm.

Other non-limiting polymerizable aromatic amine monomers include o-toluidine, m-toluidine, p-toluidine, 2,3-xylidine, 2,5-xylidine, 2,6-xylidine, 3,5-xylidine, 2-chloroaniline, 3-chloroaniline, 2-bromoaniline, and 3-bromoaniline.

The preferred sensor comprises a sensing layer consisting essentially of PAni prepared by the above-described method.

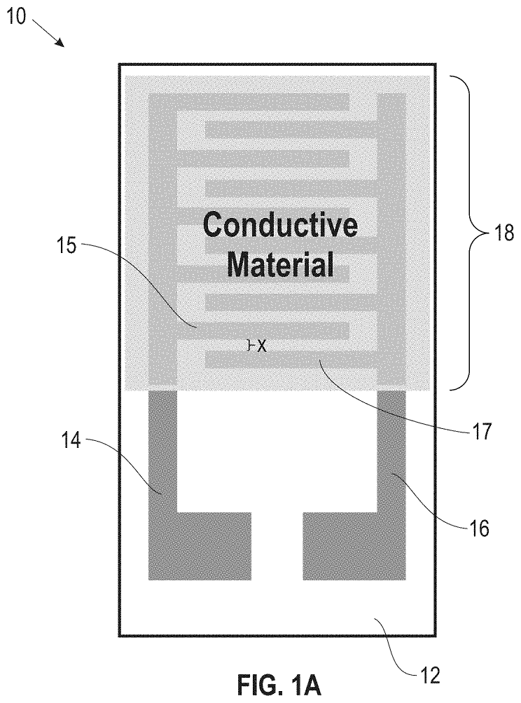

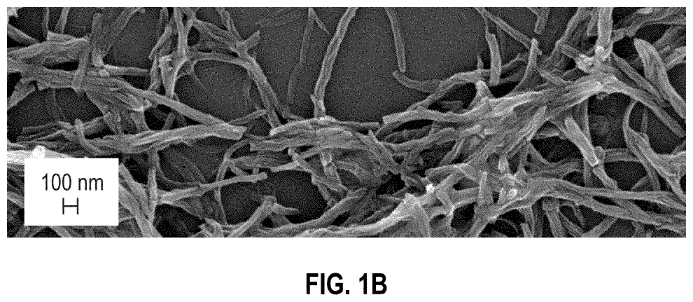

FIG. 1A is a schematic overhead drawing of a disclosed sensor 10 comprising a first electrode 14 (e.g., anode) and a second electrode 16 (e.g., cathode) disposed on a substrate 12. Substrate 12 can comprise one or more layers (e.g., glass). The electrodes preferably comprise a conductive metal selected from the group consisting of copper, aluminum, zinc, tungsten, tantalum, tin, gold, chromium, platinum, silver, nickel, and combinations thereof. First electrode 14 (e.g., cathode) and second electrode 16 (e.g., anode) are preferably comb-shaped comprising a plurality of parallel projections referred to herein as “feet”. Feet 15 of first electrode 14 are interdigitated with a plurality of feet 17 of second electrode 16, separated by gap x (e.g., 10 microns). For illustration purposes only, each of the electrodes of FIG. 1A contains 5 feet. A given electrode can comprise 2 to 10,000 feet. Gap x can be about 50 nm to about 20 microns. A given “foot” of the electrode can have a length of between 0 mm and 5mm, preferably about 1 mm, a width between 1 micron and 50 microns, preferably 15-25 microns, most preferably of about 10 microns, and a thickness of about 10-500 nm, more preferably 10-100 nm, and most preferably about 10-50 nm.

Sensor 10 comprises sensing layer 18, preferably composed of conductive PAni nanofibers formed by the above-described method. Sensing layer 18 is disposed on first electrode 14, second electrode 16, and portions of remaining top surface of substrate 12. That is, the network of nanofibers contacts both electrodes. The sensing layer containing the nanofibers can have a thickness between 1 nm and 10 μm.

The substrate preferably comprises a top surface comprising a non-conductive topcoat layer (not shown) that resides on the non-interdigitated regions of the electrodes and a portion of the substrate layer underlying the electrodes. A second portion of the top surface of substrate contains the interdigitated regions of the electrodes having no topcoat thereon (i.e., the electrode top surface is exposed to air as well as the underlying substrate layer). The liquid dispersion of conductive PAni is applied selectively to the second portion, thereby making contact with the interdigitated feet of the electrodes and underlying substrate layer. An exemplary topcoat for this purpose is CYTOP™, a fluoropolymer.

A method of forming the topcoat comprises i) forming a uniform layer of topcoat material over the top surface of a precursor substrate containing the interdigitated electrodes and ii) selectively etching (e.g., oxygen ion etching) the uniform layer of topcoat material above the interdigitated portion of the electrodes.

Sensing layer 18 is porous due to the network of fibers of the conductive material. As an example, FIG. 1B depicts a scanning electron micrograph (SEM) image of PAni fibers prepared as described above using OXONE™. The fibers have a cross-sectional diameter of about 50 nm. Sensitivity of the sensor increases with decreasing cross-sectional diameter of the fibers due to increased fiber surface area. Therefore, nano-scale fibers are preferred.

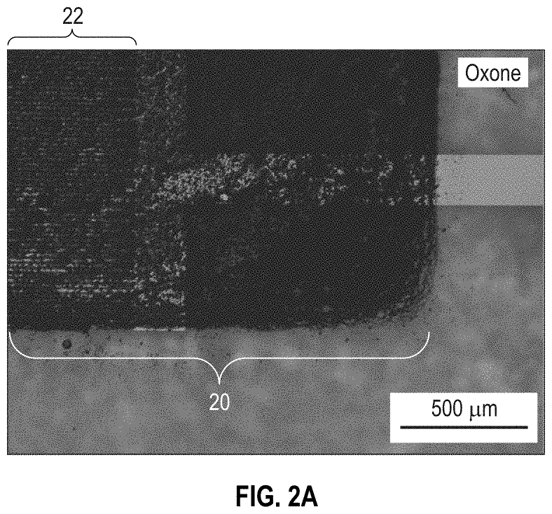

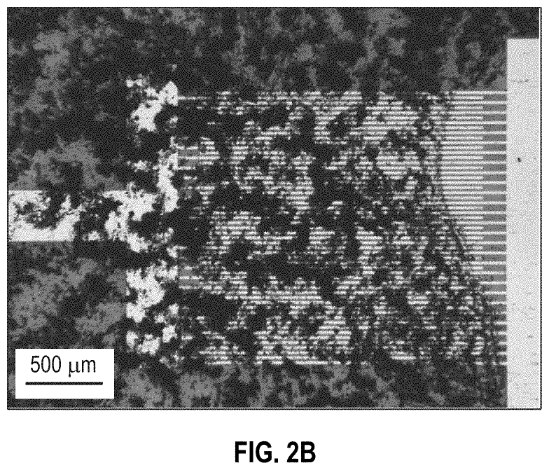

FIG. 2A is a photograph of a portion of an actual sensor showing a section of a conductive PAni layer 20 prepared by the disclosed method that was coated using a blade coater. Layer 20 is disposed on interdigitated feet 22 of two electrodes. For comparison, FIG. 2B shows a second sensor comprising a PAni layer generated using ammonium peroxydisulfate (APS), which does not yield a stable dispersion. This APS-generated PAni layer is more porous, contains less PAni, and is less sensitive compared to the PAni layer of FIG. 2A.

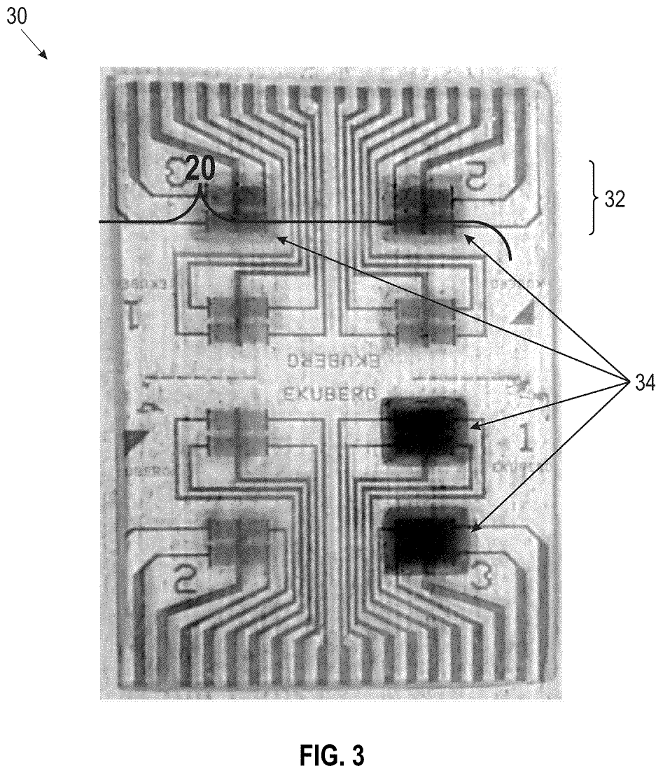

FIG. 3 is a photograph of a portion of an actual sensor chip 30 comprising 8 sensors 32, four of which contain a fibrous polyaniline layer 34 (darker shaded areas) prepared by the disclosed method. The number of sensors is for illustration purposes only and is not meant to be limited to 8. Sensor chip 30 can comprise 1 to about 100 sensors, more particularly 1 to about 50 sensors.

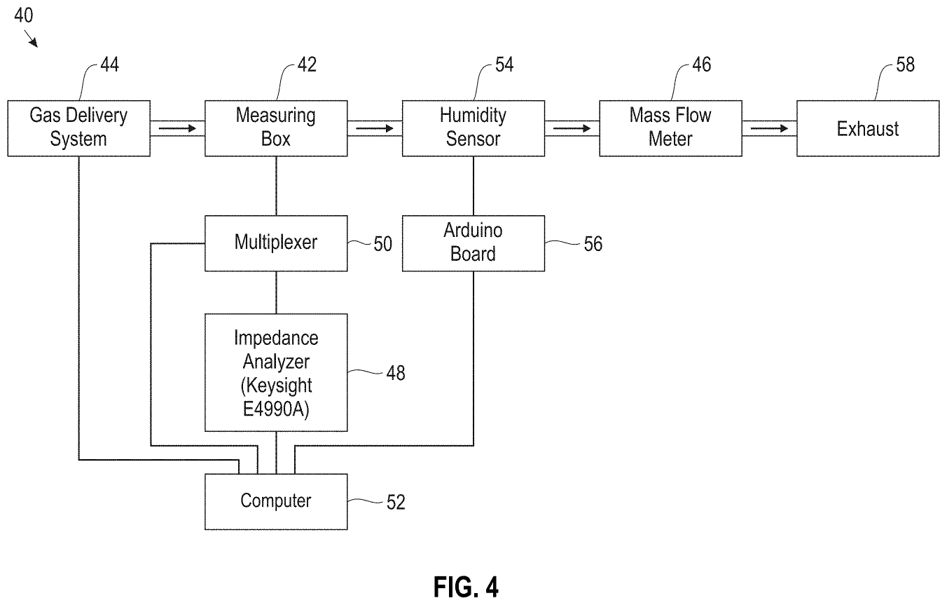

FIG. 4 is a diagram of a prototype sensing device 40 for detecting a compound in a gaseous state. The sensor chip is located in a measuring box 42, which receives a gas from a gas delivery system 44 at a given gas concentration (ppm). The gas flow rate is controlled by the gas delivery system and monitored by an in-line flow meter 46. The gas stream makes contact with the sensing layer of the sensor(s) while the sensing layer is subjected to a sinusoidal alternating current (AC) over a range of frequencies. An impedance analyzer 48 analyzes the sensing layer's electrical impedance at each of the different frequencies. The impedance analyzer is connected to the measuring box by way of a multiplexer 50. A computer 52 is used to further analyze, store, and display data from the impedance analyzer. Humidity-based adjustments are performed by way of a humidity sensor 54 in the gas stream, which is connected to the computer via a microcontroller 56 (e.g., Arduino board). The gas stream exits the device via exhaust 58.

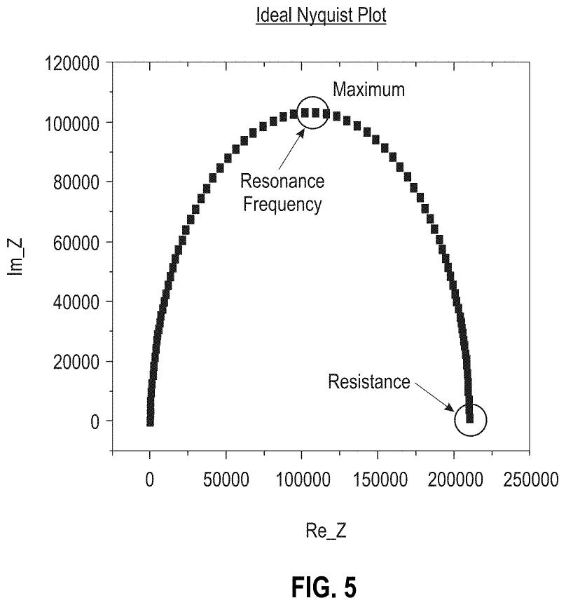

Two measurement values of the sensing layer are extracted by the impedance analyzer: i) the frequency-dependent phase shift of the impedance and ii) the change in the resistive component (ReZ) of the sensing layer. The frequency sweep per measurement can be in the range of 20 Hertz (Hz) to 20 megaHertz (MHz). The results of a measurement are transferred into the imaginary and real part of Z and displayed as a Nyquist plot, where the y-axis is −ImZ (minus the imaginary part of Z) and the x-axis is ReZ (the real part of Z). An ideal Nyquist plot corresponding to a single gas concentration is illustrated in FIG. 5. The resistance (at frequency=20 Hz) and resonance frequency of the sensing layer can be calculated from the Nyquist plot. The resistance (at frequency=20 Hz) occurs at the intersection of the Nyquist curve with the x-axis at high ReZ. The resonance frequency is the frequency corresponding to the peak of the Nyquist plot.

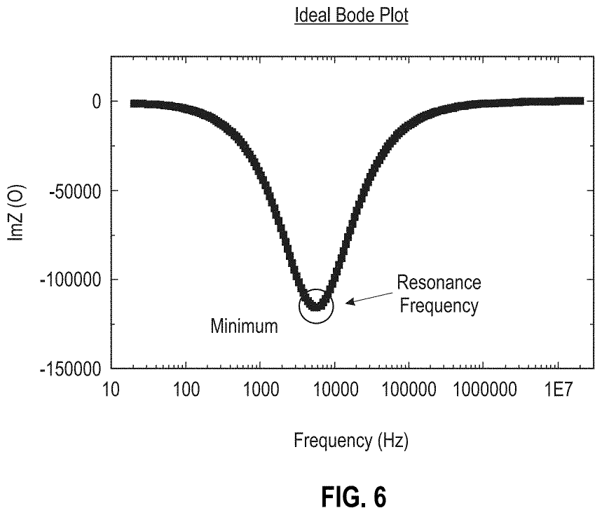

A more straightforward way of obtaining the resonance frequency is with a Bode plot shown in FIG. 6, where the x-axis is the frequency and the y-axis is ImZ. The minimum of the curve is the resonance frequency. A Bode plot typically refers to magnitude or phase versus frequency. Herein, Bode plots are extended to any variable (e.g., ImZ) plotted versus frequency.

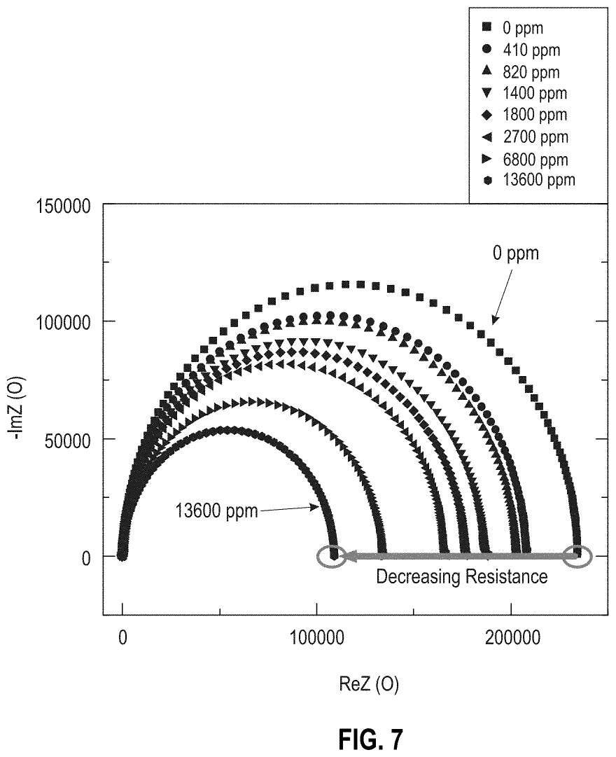

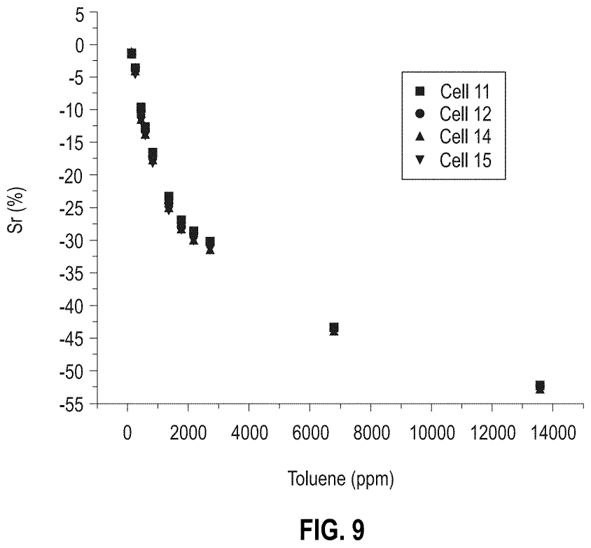

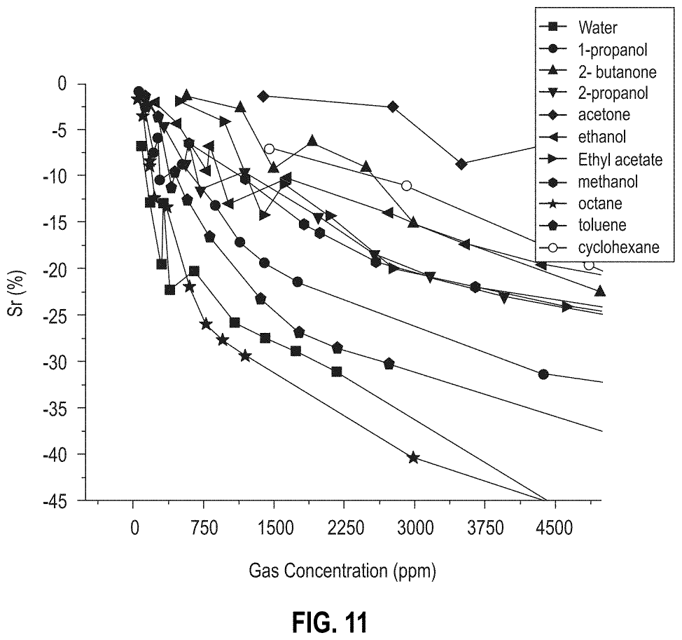

The conductivity of the sensing layer increases with increased concentration of a gaseous analyte in contact with the sensing layer. The analyte can be polar (e.g., water vapor) or non-polar (e.g., octane vapors). This is illustrated in FIGS. 7 (also FIGS. 9 and 11 described below).

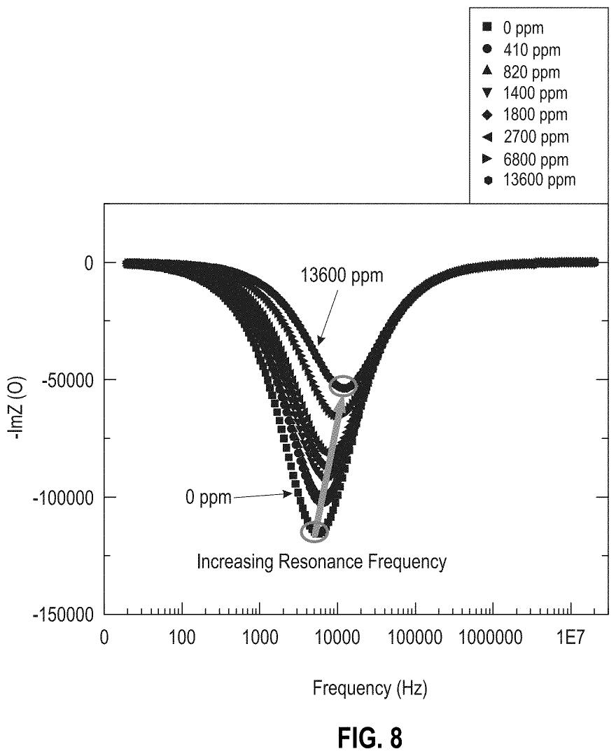

FIG. 7 shows a family of Nyquist plots obtained for different concentrations (ppm) of toluene vapor using the disclosed PAni sensor. The arrow in FIG. 7 indicates that the impedance (resistance) of the conductive PAni layer decreases with increasing toluene concentration. FIG. 8 shows the corresponding Bode plots for toluene at the different concentrations. The arrow in FIG. 8 shows the resonance frequency increases with increasing concentration of toluene.

The percent change in impedance, Sᵣ(%), and the percent change in resonance frequency, S_{f}(%), are then plotted versus the analyte concentration range. Sᵣ(%) and S_{f}(%) are defined as follows.

Sᵣ(%)=100×(R_{analyte}−R₀)/R₀

S_{f}(%)=100×(f_{analyte}−f₀)/f₀

R₀ is the impedance at 0 ppm, R_{analyte} is the impedance at a given concentration greater than 0 ppm, f₀ is the resonance frequency at 0 ppm, and f_{analyte} is the resonance frequency at a given concentration greater than 0 ppm.

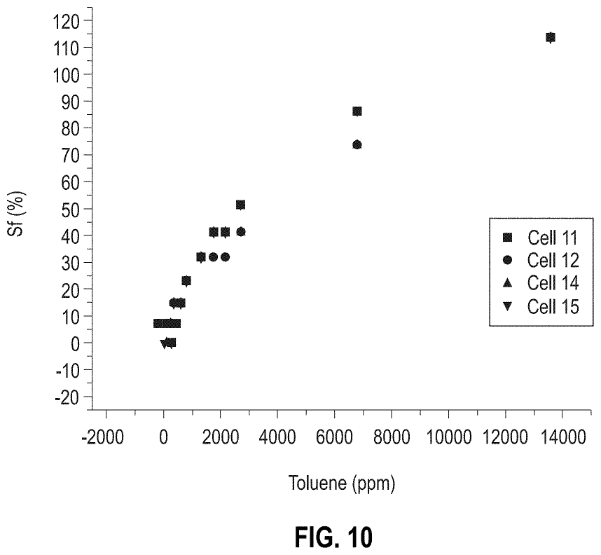

FIG. 9 and FIG. 10 are graphs showing Sᵣ(%) and S_{f}(%), respectively, as a function of toluene concentration in ppm. The reproducibility of the plots is demonstrated in each case using four iterations.

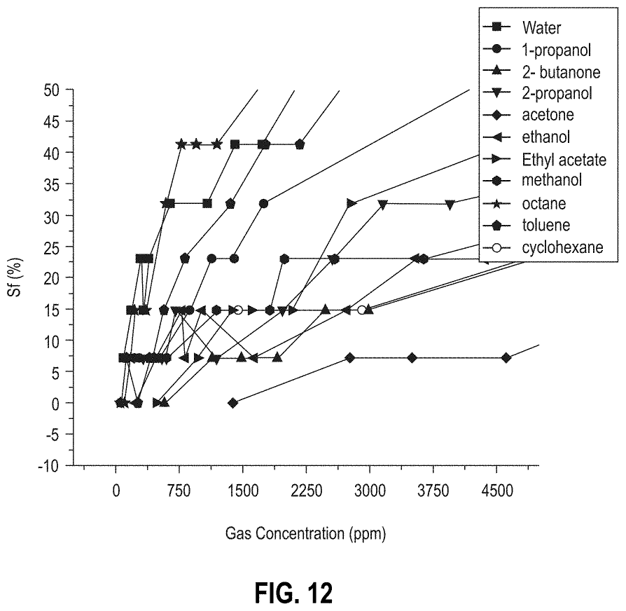

Other VOCs exhibit different Sᵣ(%) and S_{f}(%) profiles. FIG. 11 and FIG. 12 compare Sᵣ(%) and S_{f}(%), respectively, of water, 1-propanol, 2-butanone, 2-propanol, acetone, ethanol, ethyl acetate, methanol, octane, toluene, and cyclohexane. These compounds were chosen for illustration purposes only and are not meant to be limiting.

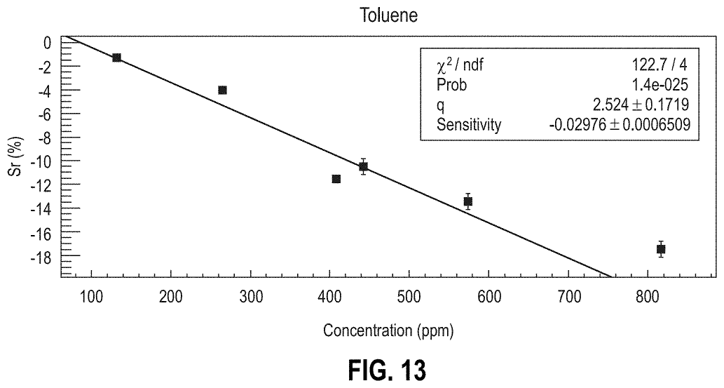

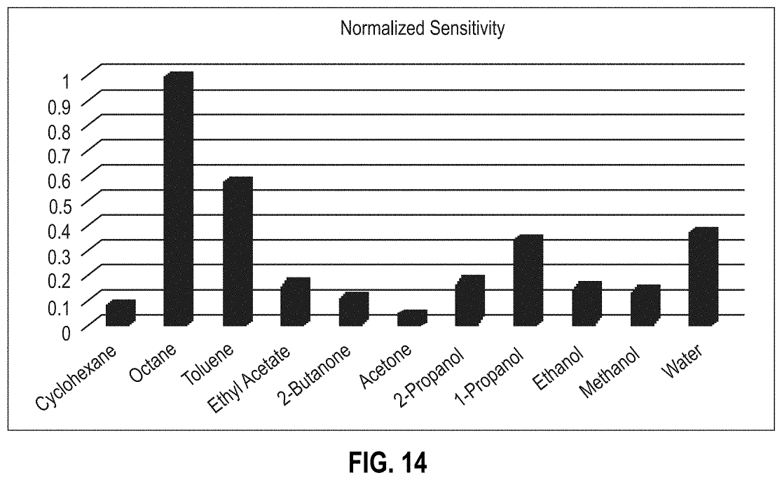

The sensitivity of the Sᵣ(%) measurement is estimated as the slope of the linear fit of the Sᵣ(%) data points at the six lowest concentrations of the gas. FIG. 13 depicts this line for toluene, which has a slope (sensitivity) of −0.02976%/ppm. The sensitivities were determined for the other above-mentioned compounds and then normalized to the less sensitive result. The normalized results are shown in FIG. 14.

The sensitivity of the disclosed prototype sensor device is generally greater than previously reported, commercial, polymer-based sensor devices based on available sensing data. For example, the response of the disclosed prototype sensor device to 200 ppm water vapor, 2000 ppm 2-propanol, 7000 ppm acetone, and 970 ppm acetone was 25 times, 89 times, 53 times, and 42 times, respectively, the response obtained for each gas using a commercially available gas sensor.

The limit of detection (LoD) in parts per million (ppm) of the disclosed sensor is also low. Table 1 lists the LoD of various analytes in ppm obtained for the disclosed prototype sensor device versus the permissible exposure limits set by the United States Department of Labor, where Cal/OSHA PEL is the California Division of Occupational Safety and Health permissible exposure limits, NIOSH REL is the National Institute for Occupational Safety and Health recommended exposure limits, TWA is time-weighted average, and STEL is the average short-term exposure limit over a 15-30 minute period of maximum exposure during a work shift. In each case, the LoD of the disclosed sensor is below both TWA limits.

TABLE 1

| | Cal/OSHA PEL | NIOSH REL | |
|---|---|---|---|
| | (as of Apr. 4, 2018) | (as of Jul. 7, 2016) | |
| | 8 h-TWA/ | Up-to-10 h-TWA/ | |
| | STEL/Ceiling | STEL/Ceiling | LoD |
| Analyte | [ppm] | [ppm] | [ppm] |
| 1-Propanol | 200/250/— | 200/250/— | 18 |
| 2-Butanone (MEK) | 200/300/— | 200/300/— | 45 |
| 2-Propanol | 400/500/— | 400/500/— | 11 |
| Acetone | 500/750/3000 | 250/—/— | 189 |
| Cyclohexane | 300/—/— | 300/—/— | 279 |
| Ethanol | 1000/—/— | 1000/—/— | 11 |
| Ethyl acetate | 400/—/— | 400/—/— | 43 |
| Methanol | 200/250/1000 | 200/250/— | 108 |
| Octane | 300/375/— | 75/385/— | 8 |
| Tetrahydrofuran | 200/250/— | 200/250/— | 46 |
| Toluene | 25/100/300 | — | 11 |
| Water | — | — | 51 |

### Computer hardware and software

The computer system for implementing the present invention can take the form of an entirely hardware embodiment, an entirely software embodiment (including firmware, resident software, microcode, etc.), or a combination of software and hardware that may all generally be referred to herein as a “circuit,” “module,” or “system.”

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

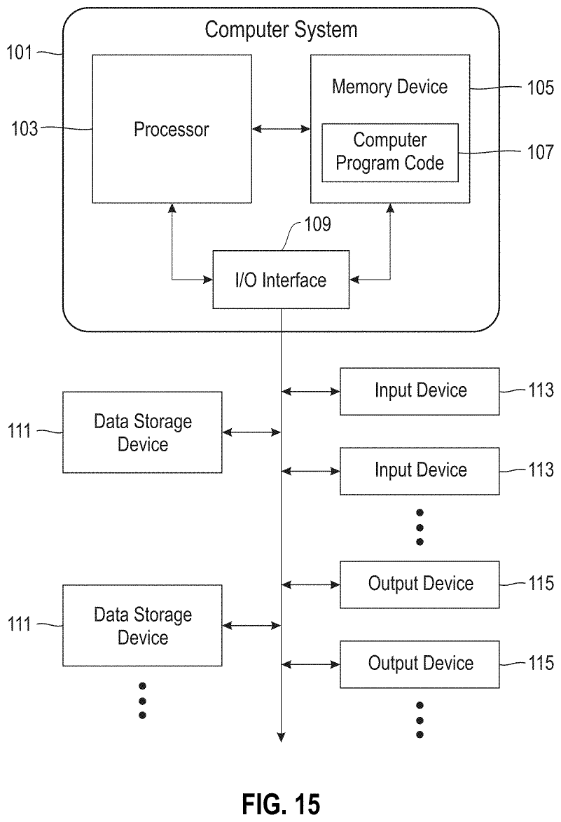

FIG. 15 is a block diagram showing an example of a structure of a computer system and computer program code that can be used to automatically and programmatically implement the processes of the invention. In FIG. 15, computer system 101 comprises a processor 103 coupled through one or more I/O Interfaces 109 to one or more hardware data storage devices 111 and one or more I/O devices 113 and 115. Hardware data storage devices 111 can contain, for example, reference sensor impedance data obtained for one or more chemical compounds.

Hardware data storage devices 111 may include, but are not limited to, magnetic tape drives, fixed or removable hard disks, optical discs, storage-equipped mobile devices, and solid-state random-access or read-only storage devices. I/O devices may comprise, but are not limited to: input devices 113, such as keyboards, scanners, handheld telecommunications devices, touch-sensitive displays, tablets, biometric readers, joysticks, trackballs, or computer mice; and output devices 115, which may comprise, but are not limited to printers, plotters, tablets, mobile telephones, displays, or sound-producing devices. Data storage devices 111, input devices 113, and output devices 115 may be located either locally or at remote sites from which they are connected to I/O Interface 109 through a network interface.

Processor 103 may also be connected to one or more memory devices 105, which may include, but are not limited to, Dynamic RAM (DRAM), Static RAM (SRAM), Programmable Read-Only Memory (PROM), Field-Programmable Gate Arrays (FPGA), Secure Digital memory cards, SIM cards, or other types of memory devices.

At least one memory device 105 contains stored computer program code 107, which is a computer program that comprises computer-executable instructions. The stored computer program code can include a program for natural-language processing that implements the disclosed methods. The data storage devices 111 may store the computer program code 107. Computer program code 107 stored in the storage devices 111 can be configured to be executed by processor 103 via the memory devices 105. Processor 103 can execute the stored computer program code 107.

Thus, the present invention discloses a process for supporting computer infrastructure, integrating, hosting, maintaining, and deploying computer-readable code into the computer system 101, wherein the code in combination with the computer system 101 is capable of automatically and programmatically implementing the disclosed processes of the invention.

Any of the components of the present invention could be created, integrated, hosted, maintained, deployed, managed, serviced, supported, etc. by a service provider. Thus, the present invention discloses a process for deploying or integrating computing infrastructure, comprising integrating computer-readable code into the computer system 101, wherein the code in combination with the computer system 101 is capable of automatically and programmatically implementing the disclosed processes of the invention

One or more data storage units 111 (or one or more additional memory devices not shown in FIG. 15) may be used as a computer-readable hardware storage device having a computer-readable program embodied therein and/or having other data stored therein, wherein the computer-readable program comprises stored computer program code 107. Generally, a computer program product (or, alternatively, an article of manufacture) of computer system 101 may comprise said computer-readable hardware storage device.

While it is understood that program code 107 may be deployed by manually loading the program code 107 directly into client, server, and proxy computers (not shown) by loading the program code 107 into a computer-readable storage medium (e.g., computer data storage device 111), program code 107 may also be automatically or semi-automatically deployed into computer system 101 by sending program code 107 to a central server (e.g., computer system 101) or to a group of central servers. Program code 107 may then be downloaded into client computers (not shown) that will execute program code 107.

Alternatively, program code 107 may be sent directly to the client computer via e-mail. Program code 107 may then either be detached to a directory on the client computer or loaded into a directory on the client computer by an e-mail option that selects a program that detaches program code 107 into the directory.

Another alternative is to send program code 107 directly to a directory on the client computer hard drive. If proxy servers are configured, the process selects the proxy server code, determines on which computers to place the proxy servers' code, transmits the proxy server code, and then installs the proxy server code on the proxy computer. Program code 107 is then transmitted to the proxy server and stored on the proxy server.

In one embodiment, program code 107 is integrated into a client, server and network environment by providing for program code 107 to coexist with software applications (not shown), operating systems (not shown) and network operating systems software (not shown) and then installing program code 107 on the clients and servers in the environment where program code 107 will function.

The first step of the aforementioned integration of code included in program code 107 is to identify any software including the network operating system (not shown), which is required by program code 107 or that works in conjunction with program code 107 and is on the clients and servers where program code 107 will be deployed. This identified software includes the network operating system, where the network operating system comprises software that enhances a basic operating system by adding networking features. Next, the software applications and version numbers are identified and compared to a list of software applications and correct version numbers that have been tested to work with program code 107. A software application that is missing or that does not match a correct version number is upgraded to the correct version.

A program instruction that passes parameters from program code 107 to a software application is checked to ensure that the instruction's parameter list matches a parameter list required by the program code 107. Conversely, a parameter passed by the software application to program code 107 is checked to ensure that the parameter matches a parameter required by program code 107. The client and server operating systems, including the network operating systems, are identified and compared to a list of operating systems, version numbers, and network software programs that have been tested to work with program code 107. An operating system, version number, or network software program that does not match an entry of the list of tested operating systems and version numbers is upgraded to the listed level on the client computers and upgraded to the listed level on the server computers.

After ensuring that the software, where program code 107 is to be deployed, is at a correct version level that has been tested to work with program code 107, the integration is completed by installing program code 107 on the clients and servers.

Embodiments of the present invention may be implemented as a method performed by a processor of a computer system, as a computer program product, as a computer system, or as a processor-performed process or service for supporting computer infrastructure.

### Utility

Non-limiting applications of the disclosed sensor include industrial emissions monitoring (e.g., detecting toxic organic compounds (e.g., formaldehyde, benzene) used in manufacturing processes), in home monitoring (e.g., detecting carbon monoxide and smoke emissions), in automotive monitoring (e.g., combustion emissions), in medical diagnostics (e.g., detecting disease from components in exhaled gases of a patient), in military applications (e.g., detecting chemical warfare agents), in agriculture (e.g., measuring effluent gases from crops as a means of determining crop health), and in food safety (e.g., measuring VOCs from food as a means of detecting contamination or spoilage).

### EXAMPLE

The following example describes the preparation of a chemical sensor comprising a sensing layer of binder-free PAni.

Aniline, OXONE™, and hydrochloric acid were purchased from commercial sources such as Sigma Aldrich. Aniline was distilled prior to use. Ultrapure water was provided by a Millipore Milli-Q Integral water purification system.

### Preparation of PAni Nanofibers

At room temperature, aniline (102 mg, 1.095 mmol, 1.00 eq.) was dissolved in 1 M aqueous hydrochloric acid (11 ml). A solution of OXONE (84 mg, 0.274 mmol, 0.25 eq.) in 1 ml water was added quickly under rapid mixing. The OXONE contained KHSO₅, KHSO₄, and K₂SO₄ in a KHSO₅:KHSO₄:K₂SO₄ molar ratio of 1:0.5:0.5. Subsequently, the stirring bar was removed and the reaction allowed to proceed undisturbed. After 40 minutes, the reaction mixture was diluted by 25 ml water and the polyaniline nanofibers were purified by four centrifugation-washing cycles. Each cycle consisted of 5 minute centrifugation at 15,000 rpm, decantation of the supernatant, adding of 35 ml water and re-suspending the sediment. After the last purification cycle, the sediment was taken into 5 ml of water and stored as a stock dispersion.

### Variations

The equivalents of OXONE can vary depending on the targeted conductivity of the polyaniline nanofibers. A co-oxidant such as sodium hypochlorite (5-7.5% chlorine, Sigma Aldrich) can be added together with the OXONE to the aniline solution in various volume ratios (e.g., 1:11 v/v). Furthermore, concentration, temperature, and reaction time are interdependent. To ensure high conversion of the aniline, the polymerization can be conducted for time period of a few minutes to a day.

### Formation of the Sensing Layer

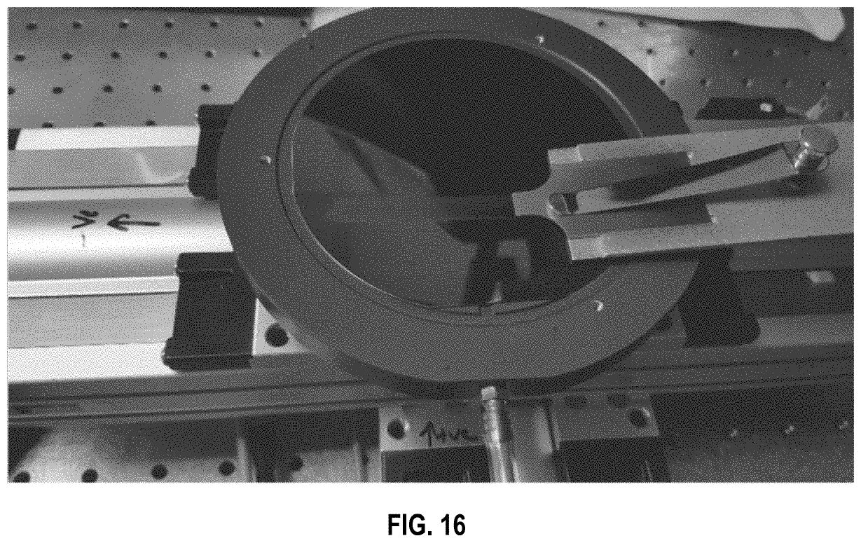

The processing of polyaniline nanofiber dispersion was performed by blade-coating. The substrate consisted of three components, a glass layer (thickness 1-2 mm), chromium/gold electrodes (thickness about 27 nm) and a CYTOP top layer (thickness about 46 nm). Areas where the polyaniline nanofibers were to be deposited on the electrodes were selectively etched by oxygen plasma to remove the CYTOP coating. Blade coating was conducted at room temperature using various volumes of the nanofiber dispersion at different dilutions, different coating speeds, blade gap heights and by single or multiple passes. The selection of the parameter values was adjusted to the targeted film thickness, but usually 2.5-7.5 μl of a 1:2-1:4 dilution was coated at a coating speed of 1 cm/s and a gap height of 10 μm in a single pass. FIG. 16 is a photograph of the coating apparatus used to coat the disclosed dispersion. The coating blade is shown above a silicon wafer after forming a narrow coating of the dispersion. The coated layer appears as a narrow hazy strip on the wafer.

### Formation of the Interdigitated Electrodes

The interdigited electrodes were fabricated by optical lithography. Specifically, the AZ5214B photoresist (from MicroChemicals GmbH) was spin-coated (300 rpm for 3 seconds and 4000 rpm for 40 seconds) on a glass substrate (from Visionteck). Then, the sample was baked at 120° C. for 1 minute, exposed for 20 seconds using a Karl Suss MJB3 mask aligner and dipped in a AZ726 developer solution for 60 seconds. Subsequently, the metal layers (3 nm Cr at a rate of 0.3 Å/s and 30 nm Au at a rate of 1.3 Å/second) were thermally evaporated and a lift-off process was carried out in acetone to obtain the interdigited electrodes array.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. When a range is used to express a possible value using two numerical limits X and Y (e.g., a concentration of X ppm to Y ppm), unless otherwise stated the value can be X, Y, or any number between X and Y.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and their practical application, and to enable others of ordinary skill in the art to understand the invention.

What is claimed is:

1. A method, comprising:
providing an initial mixture comprising a polymerizable aromatic amine monomer, an acid, and an aqueous solvent;
adding to the initial mixture an aqueous solution of KHSO₅, KHSO₄, and K₂SO₄, and optionally a co-oxidant, thereby forming a second mixture comprising an initial polymer;
removing any excess salt and monomer from the initial polymer, thereby forming fibers of an electrically conductive polyamine; and
suspending the conductive polyamine in an aqueous solvent, thereby forming a self-stabilized liquid dispersion of the conductive polyamine.

2. The method of claim 1, wherein the acid is hydrochloric acid, and the oxidative polymerization is performed at a pH between 0 and 8.

3. The method of claim 1, wherein the co-oxidant is NaOCl.

4. The method of claim 1, wherein the liquid dispersion has a pH between 0 and 8.

5. The method of claim 1, wherein the monomer is aniline and/or a substituted aniline, the substituted aniline comprising a substituent other than hydrogen at aromatic ring position 2, 3, 5 and/or 6 of aniline, and/or at the nitrogen of aniline.

6. The method of claim 1, wherein the monomer is aniline and the conductive polyamine is a polyaniline comprising at least one diimine group selected from the group consisting of: 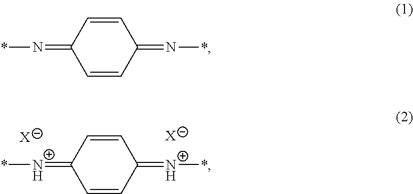
and combinations thereof, wherein each X^{⊖} is an independent negative-charged counterion.

7. A chemical sensor, comprising:
a substrate comprising a pair of interdigitated electrodes;
an electrically conductive sensing layer for sensing a chemical compound, the sensing layer disposed on the pair of interdigitated electrodes; and
an impedance analyzer in electrical communication with the sensing layer and interdigitated electrodes;
wherein
the sensing layer comprises fibers of an electrically conductive polyamine in an amount of 98-100 wt % based on total weight of the sensing layer, and
the chemical sensor uses electrochemical impedance spectroscopy to characterize a chemical compound in contact with the sensing layer.

8. The chemical sensor of claim 7, wherein the sensing layer excludes a non-conductive polymer binder for the conductive polyamine,

9. The chemical sensor of claim 7, wherein the chemical compound is in a gaseous state.

10. The chemical sensor of claim 7, wherein the sensing layer contains fibers of a conductive form of polyaniline, the fibers having an average circular diameter between 1 nm and 500 nm.

11. The chemical sensor of claim 7, wherein the sensing layer has a thickness between 1 nm and 10 μm.

12. The chemical sensor of claim 7, wherein the chemical sensor analyzes impedance of the sensing layer over a range of alternating current frequencies, generating data which are compared with reference impedance data to identify the chemical compound and determine a concentration of the chemical compound.

13. The chemical sensor of claim 11, wherein characteristics of the impedance over frequency data are used as features for machine learning.

14. A method, comprising:
detecting contact of a chemical compound with a sensing layer of a chemical sensor using electrochemical impedance spectroscopy (EIS), the chemical sensor comprising a pair of interdigitated electrodes, the sensing layer comprising an electrically conductive polyamine in an amount of 98-100 wt % based on total weight of the sensing layer, the conductive polyamine disposed on the interdigitated electrodes, the chemical sensor comprising an EIS analyzer for measuring impedance of the sensing layer at different alternating current frequencies; and
comparing the measured impedance to reference impedance data, thereby identifying the chemical compound and determining a concentration of the chemical compound.

15. The method of claim 14, wherein the chemical compound is in a gaseous state.

16. The method of claim 14, wherein said comparing the generated impedance data to reference impedance data provides a concentration of the chemical compound.

17. The method of claim 14, wherein the alternating current frequencies are in the range of 20 Hz to 20 MHz.

18. The method of claim 14, wherein the conductive polyamine is a conductive form of polyaniline.

19. A method of making polyaniline fibers, comprising:
mixing an aniline monomer, an aqueous solvent, KHSO₅, KHSO₄, and K₂SO₄ together, thereby forming an aqueous solution comprising polyaniline nanofibers, the aqueous solution having a pH value of less than 8, the method being performed at a temperature between −5° C. and 110° C.

20. The method of claim 19, wherein the KHSO₅, KHSO₄, and K₂SO₄ are used in a KHSO₅:KHSO₄:K₂SO₄ molar ratio of 1:0.5:0.5.

21. An electrochemical impedance spectroscopy sensor for volatile organic compounds, comprising:
a substrate having multiple pairs of electrodes thereon; and
conductive polymer nanofibers in contact with the electrodes, the nanofibers made using the method of claim 19.

22. The chemical sensor of claim 21, wherein the nanofibers have a diameter between 1 nm and 500 nm.

23. The chemical sensor of claim 21, wherein the thickness of the polymer nanofibers on the electrodes is in the range from 1 nm to 10 μm.

24. A method, comprising:
using the sensor of claim 21 to obtain electrochemical impedance spectroscopy measurements, thereby identifying an analyte of interest.

25. The method of claim 24, wherein the analyte is a gaseous compound or a volatile organic compound.

26. The method of claim 24, wherein the change in resonance frequency and resistance are used to identify the analyte.

27. The method of claim 24, wherein characteristics of the impedance over frequency data are used as features for a machine learning technique.
